(19) United States
(12) Patent Application Publication
(10) Pub. No.: US 2004/0076654 A1
(43) Pub. Date: Apr. 22, 2004

(54) Branched alcohol-based personal care compositions

(75) Inventors: Philip Kyle Vinson, Fairfield, OH (US); Robert Emerson Stidham, Lawrenceburg, IN (US); Daniel Stedman Connor, Cincinnati, OH (US); Jack Clinton Heffner, Cincinnati, OH (US); Thomas Anthony Cripe, Loveland, OH (US); Jeffrey John Scheibel, Loveland, OH (US); Kevin Lee Kott, New Castle Upon Tyne (GB)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/647,974

(22) Filed: Aug. 26, 2003

Related U.S. Application Data
(60) Provisional application No. 60/408,826, filed on Sep. 7, 2002.

(51) Int. Cl. A61K 7/00

(52) U.S. Cl. 424/401

(57) ABSTRACT

Personal care compositions and methods for using them are provided. More particularly, the present invention relates to branched alcohol-based (alcohol and/or its derivatives) personal care compositions and methods employing same.

### CROSS REFERENCE TO RELATED APPLICATION

This application claims priority under 35 U.S.C. §119(e) from Provisional Application Serial No. 60/408,826, filed on Sep. 7, 2002.

### FIELD OF THE INVENTION

The present invention relates to personal care compositions and methods for using them. More particularly, the present invention relates to branched alcohol-based (alcohol and/or its derivatives) personal care compositions and methods employing same.

### BACKGROUND OF THE INVENTION

Conventional personal care compositions have utilized linear and short-chain (average number of carbons in the chain being less than 14.5) branched alcohols and alcohol ethoxylates as well as other alcohol derivatives. However, conventional linear alcohols have a tendency to crystallize out of compositions at lower temperatures. To avoid the crystallization issues, the formulator has limited options, which include 1) shortening the chain length of the linear alcohol used, which often leads to issues with product odor or viscosity, due to product thinning; 2) using unsaturated oleyl alcohol which has issues with oxidative stability, color, and/or odor, and often cost and availability; and 3) using a commercially available branched alcohol of sufficient chain length to avoid odor, however, such branched alcohols are in limited supply, expensive, available only in limited in chain length (i.e., isostearyl) and in branching position (i.e. Guerbet alcohols or conventional oxo-branched alcohols that are 2-alkyl branched).

Compositions containing vernix to provide therapeutic treatment in a human, and methods for using the compositions have been described in the art (U.S. Pat. Nos. 6,333,041 and 5,989,577 to Hoath et al.). These compositions describe the use of natural and synthetic vernix for use in skin protection, wound healing, diapers, feminine protection and restoration of epidermal barrier function. The lipid component of vernix has been reported in Stewart et al., J. Invest. Dermatol, 78:291-295 (1982); Nicolaides, Lipids 6:901-905 (1972) “The Structures of the Branched Fatty Acids in the Wax Esters of Vernix Caseosa”; and Nicolaides, J. of Chromatographic Science 13:467473 (1975) “The Determination of the position Isomers of the Methyl Branched Fatty Acid Methyl Esters by Capillary GC/MS”; and Nicolaides, Lipids 11:11 “Further Studies of the Saturated Methyl Branched Fatty Acids of Vernix Caseosa Lipid”. Lipids are defined to include fats or fat-like substances, waxes, wax-esters, sterol esters, diol esters, triglycerides, free sterols and fatty acids, which have a chain length from C12 to C26 and may be linear or branched, saturated or unsaturated. The branched chain saturated fatty acids derived from vernix caseosa are shown to comprise significant levels of iso fatty acids (e.g., about 30%), anteiso fatty acids (e.g., about 10%), internal monomethyl branched fatty acids (e.g., about 15%), and dimethyl branched fatty acids (about 1 to 2%). The harvesting of natural vernix has practical limitations, which makes the use of a synthetic vernix attractive.

The invention herein provides the formulator with a convenient source of anteiso-, internal monomethyl-, and internal dimethyl-branched chain materials.

Accordingly, there is a need for an alcohol and/or an alcohol derivative for use in personal care compositions that avoids the problems with conventional alcohols and alcohol derivatives used in personal care compositions.

### SUMMARY OF THE INVENTION

The present invention fulfills the problems identified above by providing a personal care composition comprising a branched alcohol and/or its derivatives.

In one aspect of the present invention, a personal care composition comprising:

a) branched alcohols, their derivatives, and mixtures thereof, wherein the branched alcohols have the formula:

A-X

wherein X is a hydroxy moiety; and A is a hydrophobic mid-chain branched alkyl moiety comprising C₁₂ to C₂₄ total carbons having: (1) a longest linear carbon chain (Cω) attached to the X moiety in the range of from 11 to 23 carbon atoms; (2) one or more C₁-C₃ alkyl moieties branching from this longest linear carbon chain; (3) at least one of the branching alkyl moieties attached directly to a carbon of the longest linear carbon chain at a position that is within the range of position 3 carbon, counting from position 1 carbon which is attached to the X moiety, to position ω−2 carbon, the terminal carbon minus 2 carbons; and (4) the branched alcohols or their derivatives have an average total number of carbon atoms in the A moiety in the range of from about 14.5 to about 17.9 or within the range of from about 18.1 to about 21.5; and

b) a personal care adjunct; is provided.

In yet another aspect of the present invention, a method for treating a human's body comprising contacting the human's body with a personal care composition according to the present invention.

### DETAILED DESCRIPTION OF THE INVENTION

Personal Care Composition

“Personal care composition” as used herein means any composition that contacts and/or comes into contact with a human's body, including skin, hair, teeth, fingernails, and the like. Nonlimiting examples of such personal care compositions include, but are not limited to, diaper lotions, liquid dishwashing compositions, antiperspirants, deodorants, skin foundations, lipsticks, anti-dandruff compositions, conditioners, shampoos, shower gels, body washs, bath foams, hand soaps, skin anti-wrinkle compositions, niacinamide transfer compositions, suntan lotions, moisturizing cream compositions, skin care compositions and topical medicinal compositions, such as burn care compositions.

The personal care composition of the present invention may be in any physical form, such as liquid, water in oil emulsion, oil in water emulsion, multiple emulsion, microemulsion, solid, powder, bar, tablet, gel, foam, paste, mousse, granule, spray, aerosol, liquid crystal dispersion, isotropic solution, crystalline dispersion, lotion, and cream. The personal care composition may be delivered by spraying, rubbing or brushing onto a human's body. The personal care composition may be associated with a substrate or carrier, such as a wipe, a sheet, a sponge, an absorbent article, or other substrate that has the personal care composition releasably adsorbed or absorbed to the substrate.

In addition, the personal care compositions of the present invention may be incorporated into various product forms such as body care products, such as diaper lotions, antiperspirants, deodorants; cosmetics such as foundations, lipstick, make-up; skin care products, such as anti-wrinkle products, moisturizing products; suntan or sunscreen products; facial cleansing products; body wash products, such as bath foams, bath gels, shower gels, hand soap, bar soap; and haircare products, such as shampoos, conditioners, styling products, anti-dandruff products.

The personal care composition of the present invention may comprise from about 0.01% to about 40%, preferably from about 0.1% to about 20% and more preferably from about 0.1% to about 10%, by weight of the composition, of branched alcohols, their derivatives, or mixtures thereof. Unless otherwise indicated, reference to alcohols encompasses the alcohols per se as well as derivatives of such alcohols, wherein the derivatives may be any of the suitable classes of derivatives as described herein.

The personal care composition of the present invention may comprise from about 0.01% to about 80%, preferably from about 0.1% to about 40% and more preferably from 0.1% to about 20% by weight of the composition of a personal care adjunct.

The personal care composition may be in the form of an aqueous liquid.

In one embodiment, the personal care composition may further include natural or linear, or 2-alkyl branched alcohols, their derivatives, or mixtures thereof.

A. Branched Alcohol

The branched alcohol in accordance with the present invention may have the formula:

A-X

wherein X is a hydroxy moiety; and A is a hydrophobic mid-chain branched alkyl moiety comprising C₁₂ to C₂₄ total carbons having: (1) a longest linear carbon chain (Cω) attached to the X moiety in the range of from 11 to 23 carbon atoms; (2) one or more C₁-C₃ alkyl moieties branching from this longest linear carbon chain; (3) at least one of the branching alkyl moieties attached directly to a carbon of the longest linear carbon chain at a position that is within the range of position 3 carbon, counting from position 1 carbon which is attached to the X moiety, to position ω−2 carbon, the terminal carbon minus 2 carbons; and (4) the branched alcohols or their derivatives have an average total number of carbon atoms in the A moiety in the range of from about 14.5 to about 17.9 or within the range of from about 18.1 to about 21.5.

In one embodiment, the branched alcohols or their derivatives have an average total number of carbon atoms in the A moiety in the range of from about 14.5 to about 17.5 or within the range of from about 18.5 to about 21.5.

In another embodiment of the present invention, certain points of branching are preferred over other points of branching along the backbone of the hydrophobic moiety A. The formula below illustrates the mid-chain branching range (i.e., where points of branching occur), preferred mid-chain branching range, and more preferred mid-chain branching range for mono-methyl branched alkyl A moieties useful according to the present invention.

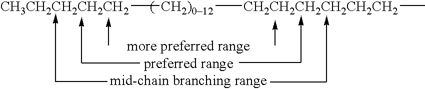

It should be noted that for the mono-methyl substituted alcohols these ranges exclude the two terminal carbon atoms of the chain and the two carbon atoms immediately adjacent to the —X group.

The formula below illustrates the mid-chain branching range, preferred mid-chain branching range, and more preferred mid-chain branching range for at least one methyl group of a di-methyl substituted alkyl A moieties useful according to the present invention.

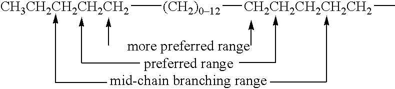

Nonlimiting examples of the C₁₆ and C₁₇ mono-methyl branched primary alcohols of the present invention may be selected from the group consisting of: 3-; 4-; 5-; 6-; 7-; 8-; 9-; 10-; 11-; 12-; 13-methyl pentadecanol; 3-; 4-; 5-; 6-; 7-; 8-; 9-; 10-; 11-; 12-; 13-; 14-methyl hexadecanol and mixtures thereof.

Nonlimiting examples of the C₁₆ and C₁₇ di-methyl branched primary alcohols of the present invention may be selected from the group consisting of: 2,3-; 2,4-; 2,5-; 2,6-; 2,7-; 2,8-; 2,9-; 2,10-; 2,11-; 2,12-methyl tetradecanols, 2,3-; 2,4-; 2,5-; 2,6-; 2,7-; 2,8-; 2,9-; 2,10-; 2,11-; 2,12-; 2,13-methyl pentadecanols and mixtures thereof.

### Synthesis Scheme (I) of Branched Alcohols

The following synthesis scheme outlines a general approach to the preparation of the mid-chain branched primary alcohol, which can then be derivatized by known methods, if desired.

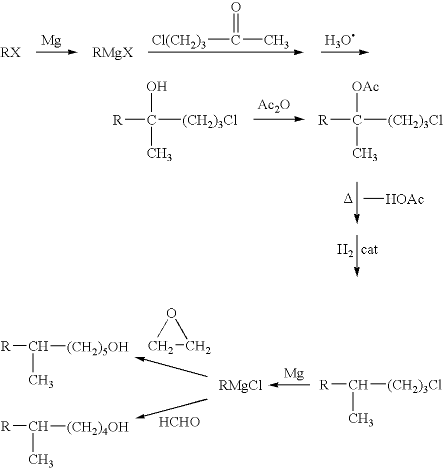

An alkyl halide is converted to a Grignard reagent, which is reacted with a haloketone. After conventional acid hydrolysis, acetylation and thermal elimination of acetic acid, an intermediate olefin is produced (not shown in the scheme) which is hydrogenated forthwith using any convenient hydrogenation catalyst such as Pd/C.

This synthesis route is favorable over others in that the branch, a 5-methyl branch in this illustration, is introduced early in the reaction sequence.

Formylation of the alkyl halide resulting from the first hydrogenation step yields alcohol product, as shown in the scheme. There is flexibility to extend the branching one additional carbon beyond that which is achieved by a single formylation. Such extension can, for example, be accomplished by reaction with ethylene oxide. See “Grignard Reactions of Nonmetallic Substances”, M. S. Kharasch and O. Reinmuth, Prentice-Hall, N.Y., 1954; J. Org. Chem., J. Cason and W. R. Winans, Vol. 15 (1950), pp 139-147; J. Org Chem., J. Cason et al., Vol. 13 (1948), pp 239-248; J. Org Chem., J. Cason et al., Vol. 14 (1949), pp 147-154; and J. Org Chem., J. Cason et al., Vol. 15 (1950), pp 135-138.

### Synthesis Scheme (II) of Branched Alcohol

The branched alcohols of the present invention may also be made, for example, by skeletally isomerizing olefins and then hydroformylating them to a skeletally isomerized primary branched alcohol.

The example below demonstrates the making of a skeletally isomerized C₁₆ olefin, which is then converted to a skeletally isomerized C₁₇ primary alcohol composition of the present invention.

About 1 liter of NEODENE® 16 olefin, a C₁₆ linear alpha.-olefin commercially available from Shell Chemical Company, is first dried and purified through alumina. The olefin is then passed through a tube furnace at about 250° C. set at a feed rate of about 1.0 ml/minute and using a nitrogen pad flowing at about 91 cc/minute. Working from the top, the tube furnace is loaded with glass wool, then about 10 ml of silicon carbide, then the catalyst, followed by 5 ml of silicon carbide, and more glass wool at the bottom. The volume of the tube furnace is about 66 ml. The reactor tube furnace has three temperature zones, with a multipoint thermocouple inserted into the tube reactor and positioned such that the temperature above, below and at three different places in the catalyst bed could be monitored. The reactor is inverted and installed the in the furnace. All three zones, including the catalyst zone, are kept at about 250° C. during the reaction and the pressure is maintained in the reactor at about 2 psig.

The amount of catalyst used is about 23.1 g, or about 53 ml by volume. The type of catalyst used to structurally isomerize the NEODENE® 16 olefin is a 1/16″ extruded and calcined H-ferrierite containing 100 ppm palladium metal.

This catalyst is prepared in accordance with example C of U.S. Pat. No. 5,510,306, reproduced in part herein for convenience. An ammonium-ferrierite having a molar silica to alumina ratio of 62:1, a surface area of 369 square meters per gram (P/Po=0.03), a soda content of 480 ppm and n-hexane sorption capacity of 7.3 g per 100 g of zeolite is used as the starting zeolite. The catalyst components are mulled using a Lancaster mix muller. The mulled catalyst material is extruded using an one inch or a 2.25 inch Bonnot pin barrel extruder.

The catalyst is prepared using 1 weight percent acetic acid and 1 weight percent citric acid. The Lancaster mix muller is loaded with 645 grams of ammonium-ferrierite with a loss of weight on ignition (LOI) of 5.4% and 91 grams of CATAPAL® D alumina (LOI of 25.7%) available from Sasol North America, Houston, Tex. The alumina is blended with the ferrierite for 5 minutes, during which time 152 milliliters of de-ionized water is added. A mixture of 6.8 grams glacial acetic acid, 7.0 grams of citric acid and 152 milliliters of de-ionized water is added slowly to the muller in order to peptize the alumina. The mixture is mulled for 10 minutes. 0.20 Grams of tetraammine palladium nitrate in 153 grams of de-ionized water are then added slowly as the mixture is mulled for a period of 5 additional minutes. Ten grams of METHOCEL® F4M hydroxypropyl methylcellulose available from Sigma, St. Louis, Mo., is added and the zeolite/alumina mixture is mulled for 15 additional minutes. The extrusion mix has a LOI of 43.5%. The 90:10 zeolite/alumina mixture is transferred to the 2.25 inch Bonnot extruder and extruded using a die plate with ⅙″ holes.

The moist extrudates are tray dried in an oven heated to 150° C. for 2 hours; then, the oven temperature is increased to 175° C. for 4 hours. After drying, the extrudates are manually broken, lengthwise. The extrudates are calcined in flowing air at 500° C. for two hours.

The olefin is passed through the reactor furnace over a 5 hours period. Samples of 36.99 g and 185.38 g are collected at about the 1 and 5 hours point, and combined for a total of about 222 g. A portion of this sample is then vacuum distilled at about 4 mmHg to obtain a predominate amount of the C₁₆ skeletally isomerized olefin by collecting distillate cuts (i) boiling at 160° C. in the pot and 85° C. at the head, and (ii) boiling at 182° C. in the pot and 75° C. at the head.

A 90 grams sample of the 110.93 grams of the skeletally isomerized olefin is then hydroformlyated using the modified oxo process. 90 grams of the skeletally isomerized olefin is reacted with hydrogen and carbon monoxide in about a 1.7:1 molar ratio in the presence of a phosphine modified cobalt catalyst at a temperature of up to about 185° C. and a pressure of about 1100 psi (7.6×10⁶ Pa) for about four and one-half hours in a nitrogen purged 300 cc autoclave. After completion of the reaction, the product is cooled to 60° C.

About 40 grams of the hydroformylated product is poured into a 100 ml flask and vacuum distilled for about 4 hours at about 4 mmHg with temperature increases from start of 89° C. to a finish temperature of 165° C. Distillate cuts of 20.14 g and 4.12 g are taken at 155° C. and 165° respectively, and combined in a 100 ml flask.

To the distillate cuts in the flask is added 0.2 g of sodium borohydride, stirred, and heated up to 90° C. over an 8 hour period to deactivate the hydroformylation catalyst and stabilize the alcohols. The distilled alcohol is washed with 90° C. water three times, dried with sodium sulfate, and filtered into a 100 ml flask. The alcohol is then vacuum distilled for about 1 more hour to distill off any remaining water. The product is then subjected to NMR analysis and sulfation to test for cold water solubility, detergency, and biodegradability.

B. Branched Alcohol Derivatives

“Branched alcohol derivative” as used herein means any material that is derived from the branched alcohols of the present invention; particularly branched alcohol esters (e.g., alcohol formate, alcohol acetates, alcohol butyrate, alcohol isobutyrates, alcohol glycolates, alcohol lactates, alcohol monomaleate, alcohol monosuccinate, alcohol monophthalate, alcohol cocoate, alcohol myristate, alcohol palmitate, alcohol stearate, alcohol oleate, alcohol bezoate, alcohol salicylate; branched dialcohol esters such as dialcohol malate, dialcohol maleate, dialcohol succinate, dialcohol adipate, dialcohol sebacate; and branched tetra-alcohols such as butane1,2,3,4-tetracarboxylate), branched alcohol alkoxylates (e.g., alcohol ethoxylates, alcohol propoxylates, alcohol mixed ethoxylates/propoxylates), branched alcohol ethers (e.g., alcohol glyceryl ether), branched carboxylic acids, branched carboxylic acid esters (e.g., mono-, di- and tri-glycerides of the branched carboxylic acids). The “branched carboxylic acids” as used herein refers to the oxidation products of the branched alcohols of the present invention.

Alcohol derivatives can have a very large variety of structures and include natural and synthetic types, saturated or unsaturated, linear-, branched- or cyclic-, aliphatic monoalcohol derivatives, diol derivatives or polyol derivatives; and aromatic or heterocyclic alcohol derivatives including natural alcohol derivatives, such as sugars; and heteroatom-functional aliphatic alcohol derivatives, such as aminoalcohol derivatives.

In a typical embodiment of the present invention, alcohol derivatives can be saturated or unsaturated, can be linear, or can have a great variety of branching types, depending on the size and position of branching moieties. The great variety of suitable alcohol derivatives can also be distinguished by their analytical characterization (e.g., by NMR), their performance properties, or the process by which they are made.

In a specific embodiment of the present invention, alcohol derivatives can be branched oxo alcohol derivatives, wherein at least 60% of said oxo alcohol derivatives comprising at least one C₁-C₃ alkyl branch on a third or higher carbon atom as counted from the hydroxyl group of the parent alcohol.

The branched alcohol derivatives herein may include branched primary saturated aliphatic acyclic oxo monoalcohol derivatives, at least 60% of these alcohol derivatives comprising at least one C₁-C₃ alkyl branch on a third or higher carbon atom counting from the alcohol derivative functional group (i.e., ester group, alkoxylate group, etc.).

1. Branched Alcohol Esters

Branched alcohol esters derived from the branched alcohols of the present invention may be made from any known process. A nonlimiting synthesis example is provided below.

Acetate ester derivative is made by a base catalyzed transesterification of a branched, fatty alcohol with ethyl acetate. Add 150 g (0.60 mol) of C14-C15 mid-chain branched alcohol of the present invention, 1-L ethyl acetate, and 13 g (0.06 mol) of 25% sodium methoxide in methanol. Let stir at room temperature overnight (17-19 hrs). Removed ethyl acetate by reduced pressure rotary evaporation. Add 1-L fresh ethyl acetate and 13 g additional 25% sodium methoxide. Let stir overnight again as described above to allow reaction to complete. Acetate ester derivative of the mid-chain branched alcohol is obtained.

The branched alcohol ester can be derived from the complete or partial esterification of the branched alcohol with a carboxylic acid. The carboxylic acid may be selected from the group consisting of: mono-, di-, tri- or tetra-carboxylic acids and mixtures thereof. The carboxylic acid may be selected from the group consisting of: succinic acid, citric acid, adipic acid, lactic acid, tartaric acid, phthallic acid, malic acid, maleic acid, glutaric acid, phosphoric acid, phosphorous acid, butane-1,2,3,4-tetracarboxylic acid, salicylic acid, alpha-hydroxy acid and mixtures thereof.

2. Branched Alcohol Alkoxylates

Branched alcohol alkoxylates derived from the branched alcohols of the present invention may be made by any known process. A nonlimiting synthesis example is provided below:

Alcohol ethoxylate derivative is made by mixing a branched, fatty alcohol with ethylene oxide gas in the presence of sodium metal. Add 350 g (1.40 mol) of C14-C15 mid-chain branched alcohol of the present invention and heat alcohol to 90° C. under a nitrogen blanket. Add 1.62 g (0.07 mol) of sodium metal and allow to react. Continue heating to 130° C. and cease nitrogen flow and add the ethylene oxide gas to the alcohol/sodium alkoxide mixture while stirring. Alcohol ethoxylate of the mid-chain branched alcohol is obtained.

The branched alcohol alkoxylate may be selected from the group consisting of: ethoxylate, propoxylate and mixtures thereof.

3. Branched Alcohol Ethers

Branched alcohol ethers derived from the branched alcohols of the present invention may be made by any known process. A nonlimiting synthesis example for making a branched alcohol ether of the present invention is as follows.

Provide 173.5 g (0.69 mol) C16-C17 mid-chain methyl branched alcohol of the present invention in 150 ml methylene chloride in an ice water bath; drip into the branched alcohol 342.3 g of 25% (in toluene) diisobutylaluminum hydride over a period of 2.75 hours. Let mix and come to room temperature, then drip in 35.7 g (0.46 mol) glycidol while keeping temperature at 30-35° C. After this exothermic reaction, let stir for 72 hours at room temperature. Chill mixture and add 324 g of aqueous potassium-sodium tartrate (Rochelle's salt) and add 200 ml methylene chloride. Place in separatory funnel and add 500 ml ethyl acetate. Take organic layer and extract 2× with water, dry with Na₂SO₄ then filter through CELITE®, available from Aldrich, Milwaukee, Wis. Chromatograph with silica gel column using 80:20 chloroform:ether to elute starting branched alcohol then us 98:2 ether:methanol to recover the glycerol ether. Obtain 28.5 g of clear, slightly yellow, somewhat viscous liquid (glycerol ether).

The branched alcohol ethers may comprise a glycerol or polyglycerol ether.

4. Branched Carboxylic Acids

Branched carboxylic acids derived from the branched alcohols of the present invention may be made by any known process. For example, the branched carboxylic acids may be made as follows: 0.5 mol of a mid-chain branched alcohol of the present invention is treated with 1.5 moles of 30% hydrogen peroxide, 0.01 mol of sodium tungstate, 0.02 mol of tricaprylmethylammonium chloride, and 0.002 mol sulfuric acid. Heat with stirring to 80° C. for 6 hours. Cool and separate layers. Dissolve organic layer into 250 ml hexane. Wash two times with 200 ml each of saturated bisulfite solution. Rotary evaporate. Recover 96 grams of yellow liquid. Analysis using IR/TLC (infrared/thin layer chromatography) shows high conversion to acid.

5. Branched Carboxylic Acid Esters

Branched carboxylic acid esters derived from the branched carboxylic acids of the present invention may be made by any known process. For example, the branched carboxylic acid ester may be made as follows: Three (3) moles of a branched carboxylic acid of the present invention is mixed with 1 mol of glycerin and 10 grams of AMBERLYST® 15 (Rohm & Haas). The mixture is heated under vacuum with stirring to 95 deg C. for 6 hours. The product is cooled and the AMBERLYST® 15 is separated by filtration.

The branched carboxylic acid ester may be derived from the complete or partial esterification of a mono-, di-, tri- or polyhydric alcohol with the branched carboxylic acid of the present invention.

The mono-hydric alcohol may be selected from the group consisting of:

a) branched alcohols having the formula:

A-X

wherein X is a hydroxy moiety; and A is a hydrophobic mid-chain branched alkyl moiety comprising C₁₂ to C₂₄ total carbons having: (1) a longest linear carbon chain (Cω) attached to the X moiety in the range of from 11 to 23 carbon atoms; (2) one or more C₁-C₃ alkyl moieties branching from this longest linear carbon chain; (3) at least one of the branching alkyl moieties attached directly to a carbon of the longest linear carbon chain at a position that is within the range of position 3 carbon, counting from position 1 carbon which is attached to the X moiety, to position ω−2 carbon, the terminal carbon minus 2 carbons; and (4) the branched alcohols or their derivatives have an average total number of carbon atoms in the A moiety in the range of from about 14.5 to about 17.9 or within the range of from about 18.1 to about 21.5;

b) C1 to C30 linear alcohols;

c) C4 to C30 2-alkyl branched alcohols;

d) isopropyl alcohol;

e) cholesterol; and

f) mixtures thereof.

The C1 to C30 linear alcohols may be selected from the group consisting of: methanol, ethanol, hexanol, decanol, dodecanol, hexadecanol, lauryl alcohol, cocoyl alcohol, myristyl alcohol, cetyl alcohol, stearyl alcohol, isostearyl alcohol, tallow alcohol, oleyl alcohol, behenyl alcohol, euricyl alcohol and mixtures thereof.

The C4 to C30 2-alkyl branched alcohols may be selected from the group consisting of: Guerbet alcohols, aldol alcohols, oxo alcohols and mixtures thereof.

Nonlimiting examples of the linear alcohols include methanol, ethanol, propanol, hexanol, decanol, dodecanol, hexadecanol and the like. Nonlimiting examples of the 2-alkyl branched alcohols include isopropyl alcohol, the Guerbet alcohols such as the 2-ethyl-1-hexanol, 2-butyl-1-octanol, and those sold under the tradename ISOFOL® (Sasol) and the like, and oxo alcohols, e.g., those sold under the tradenames LIAL® (Sasol), ISALCHEM® (Sasol), NEODOL® (Shell) and the like.

The dihydric alcohol may be selected from the group consisting of: ethylene glycol, propylene-1,2-diol, propylene-1,3-diol, butane-1,2-diol, butane-1,4-diol, hexane-1,2-diol, hexane-1,6-diol and mixtures thereof.

Nonlimiting examples of linear and 2-alkyl branched C2 to C30 diols include 1,2-ethanediol, 1,2-propanediol, 1,3-propanediol, 1,2-hexanediol, 1,2-dodecanediol, 1,6-hexanediol, 2-ethyl-1,6-hexanediol, and the like.

The tri- or polyhydric alcohols may be selected from the group consisting of: glycerol, diglycerol, xylitol, sorbitol, mannitol, sucrose and mixtures thereof.

Nonlimiting examples of the linear and 2-alkyl branched C3-C30 triols include glycerol, 1,2,3-hexanetriol, 2-methyl-1,3,5-decanetriol and the like.

In addition to the mono-, di-, tri- and polyhydric alcohols, monosaccharides may be used. The monosaccharides are carbohydrates that cannot be hydrolyzed to simpler compounds. The monosaccharides include the ketose and aldose families of compounds including those of varying carbon number, e.g., triose, tetrose, pentose, hexose, and the like. Here, the monosaccharides are also intended to include the hydrogenated forms of these reducing sugars. Nonlimiting examples of monosaccharides include glucose, glucitol, fructose, mannose, mannitol, galactose, arabinose, ribitol, gulose, xylose, erythrose, threose, lyxose, xylitol, glycerol, and the like. Nonlimiting examples include maltose, sucrose, cellobiose and lactose. Similarly, the trisaccharides include all carbohydrates made up of three monosaccharide units.

B. Personal Care Adjuncts

Nonlimiting examples of personal care adjuncts for use in the personal care compositions of the present invention include, but are not limited to, aesthetic agents and other active agents. For example, the compositions may include absorbents, abrasives, anticaking agents, antifoaming agents, antimicrobial agents (such as benzoyl peroxide, erythromycin, tetracycline, clindamycin, azelaic acid, and sulfur resorcinol), binders, biological additives, buffering agents, bulking agents, chemical additives, cosmetic biocides, conditioning agents, deposition polymers, cationic polymers, denaturants, cosmetic astringents, drug astringents, external analgesics, film formers, plasticizers, preservatives, preservative enhancers, propellants, reducing agents, additional skin-conditioning agents, skin penetration enhancing agents, skin protectants, solvents, suspending agents, emulsifiers, nonionic surfactants, anionic surfactants, cationic surfactants, zwitterionic surfactants, amphoteric surfactants, Gemini surfactants, hydrotropes, thickening agents, solubilizing agents, sunscreens, sunblocks, ultraviolet light absorbers or scattering agents, sunless tanning agents, antioxidants, radical scavengers, chelating agents, sequestrants, anti-acne agents, anti-inflammatory agents, anti-androgens (such as pregnenalone and its derivatives, hops extract, oxygenated alkyl substituted bicyclo alkanes like ethoxyhexyl-bicyclo octanones, and oleanolic acid), depilation agents, desquamation agents/exfoliants, organic hydroxy acids, vitamins and derivatives thereof, and natural extracts, humectants, anti-static agents, sdiluents, emollients (such as polyisobutylene, mineral oil, petrolatum and isocetyl stearyl stearate), pearlescent aids, foam boosters, pediculocides, pH adjusting agents, proteins; and aesthetic components, such as perfumes, colorants, pigments, dyes, opacifying agents, essential oils, skin sensates, astringents, skin soothing agents, skin healing agents and the like, nonlimiting examples of these aesthetic components include panthenol and derivatives (e.g. ethyl panthenol), pantothenic acid and its derivatives, clove oil, menthol, camphor, eucalyptus oil, eugenol, menthyl lactate, witch hazel distillate, allantoin, bisabalol, dipotassium glycyrrhizinate and the like, sunscreens, thickeners, vitamins and derivatives thereof (e.g., ascorbic acid, vitamin B₃, vitamin E, tocopheryl acetate, retinoic acid, retinol, retinoids, and the like), water and viscosity adjusting agents. This list of optional components is not meant to be exclusive, and other optional components can be used. Such other materials are known in the art. Nonexclusive examples of such materials are described in Harry's Cosmeticology, 7th Ed., Harry & Wilkinson (Hill Publishers, London 1982); in Pharmaceutical Dosage Forms—Disperse Systems; Lieberman, Rieger & Banker, Vols. 1 (1988) & 2 (1989); Marcel Decker, Inc.; in The Chemistry and Manufacture of Cosmetics, 2nd. Ed., deNavarre (Van Nostrand 1962-1965); and in The Handbook of Cosmetic Science and Technology, 1st Ed. Knowlton & Pearce (Elsevier 1993).

Suitable anti-inflammatory agents include specific steroidal anti-inflammatory agents, include but are not limited to, corticosteroids such as hydrocortisone; and specific non-steroidal anti-inflammatory agents include, but are not limited to 1) the oxicams, such as piroxicam; 2) the salicylates, such as aspirin; 3) the acetic acid derivatives, such as ketorolac; 4) the fenamates, such as flufenamic and tolfenamic acids; 5) the propionic acid derivatives, such as ibuprofen and naproxen; and 6) the pyrazoles, such as phenylbutazone, oxyphenbutazone, feprazone, azapropazone, and trimethazone. Mixtures of these agents may also be employed, as well as the acceptable salts and esters of these agents. “Natural” anti-inflammatory agents are useful and may be obtained as an extract from natural sources (e.g., plants, fungi, by-products of microorganisms). Nonlimiting examples include candelilla wax, aloe vera, kola extract, chamomile, and sea whip extract.

A variety of water-miscible liquids such as lower alkanols, diols, other polyols, ethers, amines, and the like may be used as part of an aqueous liquid carrier as co-solvents.

Preferred hydrotropes for use herein are sodium, potassium, calcium and ammonium cumene sulfonate; sodium, potassium, calcium and ammonium xylene sulfonate; sodium, potassium, calcium and ammonium toluene sulfonate; and mixtures thereof

Anti-oxidants/radical scavengers such as ascorbic acid (vitamin C) and its salts, ascorbyl esters of fatty acids, ascorbic acid derivatives (e.g., magnesium ascorbyl phosphate), tocopherol (vitamin E), tocopherol sorbate, other esters of tocopherol, butylated hydroxy benzoic acids and their salts, tea extracts, grape skin/seed extracts, melanin, and rosemary extracts may be used.

The compositions of the present invention may also contain a retinoid, which aids in regulating skin condition, especially in therapeutically regulating signs of skin aging. As used herein, “retinoid” includes all natural and synthetic analogs of Vitamin A or retinol-like compounds. Preferred retinoids are retinol, tocopheryl-retinoate, retinyl palmitate, retinyl acetate, retinyl proprionate, retinal and combinations thereof.

A wide variety of sunscreening or sunblocking agents are suitable for use herein. Non-limiting examples include the metal oxides such as zinc oxide and titanium dioxide, butylmethoxydibenzoylmethane, 2-ethylhexyl-p-methoxycinnamate, phenyl benzimidazole sulfonic acid, and octocrylene. Sagarin, et al., at Chapter VIII, pages 189 et seq., of Cosmetics Science and Technology (1972), discloses numerous suitable agents.

As used herein, “chelating agent” means an active agent capable of removing a metal ion from a system by forming a complex so that the metal ion cannot readily participate in or catalyze chemical reactions. Nonlimiting examples of chelators useful in compositions of the subject invention are fiurildioxime, finrilmonoxime, diethylenetriamine pentaacetic acid, ethylene diamine tetraacetic acid, and derivatives thereof.

Compositions of the present invention may also comprise an organic hydroxy acid. Non-limiting examples of suitable hydroxy acids include salicylic acid, glycolic acid, lactic acid, 5 octanoyl salicylic acid, hydroxyoctanoic acid, hydroxycaprylic acid, and lanolin fatty acids.

A variety of desquamation agents are known in the art and are suitable for use herein, including but not limited to the organic hydroxy agents described above, zwitterionic surfactants such as cetyl betaine, and mixtures thereof.

A nonlimiting example of a depilation agent for use herein includes N-acetyl-L-cysteine.

Non-limiting examples of suitable skin lightening agents for use herein include kojic acid, arbutin, ascorbic acid and derivatives thereof, e.g., magnesium ascorbyl phosphate, and vitamin B3.

The compositions of the present invention may further comprise a zinc salt. Non-limiting examples of zinc salts include zinc citrate, zinc oxide, zinc chloride, zinc acetate, zinc stearate, zinc sulfate, and mixtures thereof.

The compositions of the present invention may further comprise a humectant, moisturizing agent or other skin conditioning agent. These materials include hydroscopic agents such as guanidine and urea; alpha-hydroxy acids such as glycolic acid and glycolate salts and lactic acid and lactate salts (e.g. ammonium and quaternary alkyl ammonium) and the like; alpha-keto acids such as pyruvic acid and the like; pyrrolidone carboxylic acid; betaine; amino acids such as serine and alanine and the like; aloe vera in any of its variety of forms (e.g., aloe vera gel); polyhydroxy alcohols such as sorbitol, mannitol, glycerol, glycerol monopropoxylate, diglycerol, triglycerol, butanetriol, hexanetriol, propylene glycol, butylene glycol, hexylene glycol and the like; polyethylene glycols; sugars and starches; sugar and starch derivatives such as glucose, fructose, and alkoxylated glucose; hyaluronic acid; lactamide monoethanolamine; acetamide monoethanolamine; sucrose polyesters of fatty acids (e.g., sucrose polycottonseedate); petrolatum; silicones; lanolin and lanolin esters; methyl isosterate and ethyl isostearate; cetyl ricinoleate; sterols (e.g., cholesterol); free fatty acids (e.g., C6-C22); C1-C22 triglycerides and natural precursors (e.g., soy bean); C1-C22 alkyl zwitterionic surfactants (e.g., LONZAINE® 16SP from Lonza Chemical Co.); lipophilic calcium chelators such as salicylic acid and derivatives; panthenol and derivatives; salts thereof and mixtures thereof.

The personal care compositions of the present invention can additionally comprise a safe and effective amount of an antidandruff agent. Non-limiting examples include sulfur, octopirox, selenium sulfide, ketoconazole and pyridinethione salts in solution and platelet forms.

Suitable electrolytes include mono-, di- and trivalent inorganic salts as well as organic salts. Suitable salts include, but are not limited to, phosphates, sulfates, nitrates, citrates and halides. The counter ions of such salts can be, but are not limited to, sodium, potassium, ammonium, magnesium or other mono-, di- and tri- valent cation.

The compositions of the present invention may also include an extract obtained by suitable physical and/or chemical isolation from natural sources (e.g., plants, fungi, by-products of microorganisms), including those known in the topical personal care art. Such extracts include plant and fungal extracts such as extracts of yeast, rice bran, and of the plant Centella Asiatica. Natural extracts of Centella Asiatica are preferred and are commercially available from MMP, Inc. of Plainfield, N.J. under the trade name(s) Centella Asiatica® E.P.C.A. (“Extract Purified of Centella asiatica”) and Genines Amel®. Genines amel is the purer form of the extract.

Compounds which are known to stimulate the production of collagen can also be used in the present invention. Such compounds include estrogens (e.g., estradiol, estriol, estrone) and estrogen mimics, vitamin D and precursors or derivatives (e.g., ergosterol, 7-dehydrocholesterol, vitamin D2, vitamin D3, calcitriol, calcipotriene, etc.), Factor X (kinetin), Factor Z (zeatin), n-methyl taurine, dipalmitoyl hydroxyproline, palrmitoyl hydroxy wheat protein, biopeptide CL, (palmitoyl glycylhistidyl-lysine), ASC III (Amplifier of Synthesis of Collagen III, E. Merck, Germany), and beta glucan.

The compositions hereof can also include natural ceramides or the like, for example, ceramide 1-6.

The compositions can also contain an oil absorbent such as are known in the art, e.g. clays (e.g. bentonite) and polymeric absorbents (e.g., MICROSPONGES® 5647 and POLYTRAP®, both commercially available from Advanced Polymer Systems, Inc. of Redwood City, Calif., USA. MICROSPONGES® 5647 is a polymer mixture derived from styrene, methyl methacrylate, and hydrogel acrylate/methacrylate.

Silica is also known as silicon dioxide or silicic anhydride. Silica is a material which can be represented by the chemical formula SiO.sub.2. See, The Merck Index, tenth edition, 1983, entry 8329, page 1220. A variety of different types of silicas which are useful herein, are known including fumed or arced silica, precipitated silica, silica gel, amorphous silica, and silica sols and colloids.

Other non-limiting examples of additional components useful herein include the following: water-soluble vitamins and derivatives thereof [e.g., vitamin C]; polyethyleneglycols and polypropyleneglycols; polymers for aiding the film-forming properties and substantivity of the composition (such as a copolymer of eicosene and vinyl pyrrolidone, an example of which is available from GAF Chemical Corporation as GANEX®.RTM. V-220). Also useful are crosslinked and noncrosslinked nonionic and cationic polyacrylamides (e.g., SALCARE® SC92 which has the CTFA designation polyquaternium 32 and mineral oil; and SALCARE® SC 95 which has the CTFA designation polyquaternium 37, mineral oil and PPG-1 trideceth-6; and the nonionic Seppi-Gel polyacrylamides, available from Seppic Corporation). Also useful are crosslinked and uncrosslinked carboxylic acid polymers and copolymers such as those containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol (examples useful herein include the carbomers, which are homopolymers of acrylic acid crosslinked with allyl ethers of sucrose or pentaerytritol and which are available as the CARBOPOL® RTM. 900 series from B. F. Goodrich, and copolymers of C10-C30 alkyl acrylates with one or more monomers of acrylic acid, methacrylic acid, or one of their short chain (i.e.,C.sub.1-4 alcohol) esters, wherein the crosslinking agent is an allyl ether of sucrose or pentaerytritol, these copolymers being known as acrylates/C10-30 alkyl acrylate crosspolymers and are commercially available as CARBOPO1®.RTM. 1342, PEMULEN® TR-1, and PEMULEN® TR-2, from B.F. Goodrich). These carboxylic acid polymers and copolymers are more fully described in U.S. Pat. No. 5,087,4415, to Haffey et al., issued Feb. 11, 1992; U.S. Pat. No. 4,509,949, to Huang et al., issued Apr. 5, 1985; U.S. Pat. No. 2,798,053, to Brown, issued Jul. 2, 1957. See also, CTFA International Cosmetic Ingredient Dictionary, fourth edition, 1991, pp. 12 and 80.

All documents cited are, in relevant part, incorporated herein by reference; the citation of any document is not to be construed as an admission that it is prior art with respect to the present invention.

While particular embodiments of the present invention have been illustrated and described, it would be apparent to those skilled in the art that various other changes and modifications can be made without departing from the spirit and scope of the invention. It is therefore intended to cover in the appended claims all such changes and modifications that are within the scope of this invention.

What is claimed is:

1. A personal care composition comprising:
a) branched alcohols, their derivatives, and mixtures thereof, wherein the branched alcohols have the formula:
A-X
wherein X is a hydroxy moiety; and A is a hydrophobic mid-chain branched alkyl moiety comprising C₁₂ to C₂₄ total carbons having: (1) a longest linear carbon chain (Cω) attached to the X moiety in the range of from 11 to 23 carbon atoms; (2) one or more C₁-C₃ alkyl moieties branching from this longest linear carbon chain; (3) at least one of the branching alkyl moieties attached directly to a carbon of the longest linear carbon chain at a position that is within the range of position 3 carbon, counting from position 1 carbon which is attached to the X moiety, to position ω−2 carbon, the terminal carbon minus 2 carbons; and (4) the branched alcohols or their derivatives have an average total number of carbon atoms in the A moiety in the range of from about 14.5 to about 17.9 or within the range of from about 18.1 to about 21.5; and
b) a personal care adjunct.

2. The personal care composition according to claim 1 wherein the A moiety has an average total number of carbon atoms within the range of greater than 14.5 to about 17.5 or within the range of from about 18.5 to about 21.5.

3. The personal care composition according to claim 1 wherein the branched alcohol derivatives are selected from the group consisting of branched alcohol esters, branched alcohol alkoxylates, branched alcohol ethers, branched carboxylic acids, branched carboxylic acid esters and mixtures thereof.

4. The personal care composition according to claim 3 wherein the branched alcohol ester is derived from complete or partial esterification of the branched alcohol with a carboxylic acid selected from the group consisting of mono-, di-, tri- or tetra-carboxylic acids and mixtures thereof.

5. The personal care composition according to claim 4 wherein the carboxylic acid is selected from the group consisting of succinic acid, citric acid, adipic acid, lactic acid, tartaric acid, phthallic acid, malic acid, maleic acid, glutaric acid, phosphoric acid, phosphorous acid, butane-1,2,3,4-tetracarboxylic acid, salicylic acid, alpha-hydroxy acid and mixtures thereof.

6. The personal care composition according to claim 3 wherein the personal care composition comprises a branched alcohol alkoxylate selected from the group consisting of ethoxylate, propoxylate and mixtures thereof.

7. The personal care composition according to claim 3 wherein the personal care composition comprises a branched alcohol ether selected from the group consisting of glycerol, polyglycerol ether and mixtures thereof.

8. The personal care composition according to claim 3 wherein the personal care composition comprises a branched carboxylic acid.

9. The personal care composition according to claim 3 wherein the personal care composition comprises a branched carboxylic acid derivative selected from the group consisting of esters, amides and mixtures thereof.

10. The personal care composition according to claim 9 wherein the personal care composition comprises a branched carboxylic acid ester derived from the complete or partial esterification of a mono-, di-, tri- or polyhydric alcohol with a branched carboxylic acid.

11. The personal care composition according to claim 10 wherein the monohydric alcohol is selected from the group consisting of:
a) branched alcohols having the formula:
A-X
wherein X is a hydroxy moiety; and A is a hydrophobic mid-chain branched alkyl moiety comprising C₁₂ to C₂₄ total carbons having: (1) a longest linear carbon chain (Cω) attached to the X moiety in the range of from 11 to 23 carbon atoms; (2) one or more C₁-C₃ alkyl moieties branching from this longest linear carbon chain; (3) at least one of the branching alkyl moieties attached directly to a carbon of the longest linear carbon chain at a position that is within the range of position 3 carbon, counting from position 1 carbon which is attached to the X moiety, to position ω−2 carbon, the terminal carbon minus 2 carbons; and (4) the branched alcohols or their derivatives have an average total number of carbon atoms in the A moiety in the range of from about 14.5 to about 17.9 or within the range of from about 18.1 to about 21.5;
b) C1 to C30 linear alcohols;
c) C4 to C30 2-alkyl branched alcohols;
d) isopropyl alcohol;
e) cholesterol; and
f) mixtures thereof.

12. The personal care composition according to claim 11 wherein the C1 to C30 linear alcohols are selected from the group consisting of methanol, ethanol, hexanol, decanol, dodecanol, hexadecanol, lauryl alcohol, cocoyl alcohol, myristyl alcohol, cetyl alcohol, stearyl alcohol, isostearyl alcohol, tallow alcohol, oleyl alcohol, behenyl alcohol, erucyl alcohol and mixtures thereof.

13. The personal care composition according to claim 11 wherein the C4 to C30 2-alkyl branched alcohols are selected from the group consisting of Guerbet alcohols, aldol alcohols, oxo alcohols and mixtures thereof.

14. The personal care composition according to claim 10 wherein the dihydric alcohol is selected from the group consisting of ethylene glycol, propylene-1,2-diol, propylene-1,3-diol, butane-1,2-diol, butane-1,4-diol, hexane-1,2-diol, hexane-1,6-diol and mixtures thereof.

15. The personal care composition according to claim 10 wherein the tri- or polyhydric alcohols are selected from the group consisting of glycerol, diglycerol, xylitol, sorbitol, mannitol, sucrose and mixtures thereof.

16. The personal care composition according to claim 1 wherein the personal care composition further comprises natural or linear or 2-alkyl branched alcohols, their derivatives, or mixtures thereof.

17. The personal care composition according to claim 1 wherein the personal care composition comprises from about 0.01% to about 40% by weight of branched alcohols, their derivatives or mixtures thereof; and from about 0.01% to about 80% by weight of the personal care adjunct.

18. The personal care composition according to claim 1 wherein the personal care adjunct is selected from the group consisting of: absorbents, abrasives, anticaking agents, antifoaming agents, antimicrobial agents, binders, biological additives, buffering agents, bulking agents, chemical additives, cosmetic biocides, conditioning agents, deposition polymers, cationic polymers, denaturants, cosmetic astringents, drug astringents, external analgesics, film formers, plasticizers, preservatives, preservative enhancers, propellants, reducing agents, other skin-conditioning agents, skin penetration enhancing agents, skin protectants, solvents, suspending agents, emulsifiers, nonionic surfactants, anionic surfactants, cationic surfactants, zwitterionic surfactants, amphoteric surfactants, Gemini surfactants, hydrotropes, thickening agents, solubilizing agents, sunscreens, sunblocks, ultraviolet light absorbers or scattering agents, sunless tanning agents, antioxidants, radical scavengers, chelating agents, sequestrants, anti-acne agents, anti-inflammatory agents, anti-androgens, depilation agents, desquamation agents/exfoliants, organic hydroxy acids, vitamins and derivatives thereof, natural extracts, humectants, anti-static agents, diluents, emollients, pearlescent aids, foam boosters, pediculocides, pH adjusting agents, proteins, perfumes, colorants, pigments, dyes, opacifying agents, essential oils, skin sensates, skin soothing agents, skin healing agents, viscosity adjusting agents, water, fillers, inerts and mixtures thereof.

19. The personal care composition according to claim 1 wherein the personal care composition is in the form of a diaper lotion, an antiperspirant, a deodorant, a foundation, a lipstick, an anti-dandruff composition, a conditioner, a shampoo, a shower gel, a body wash, a bath foam, a hand soap, an anti-wrinkle composition, a niacinamide transfer composition, a suntan lotions, a moisturizing cream composition, a skin care composition, a topical medicinal composition, or an insect repellant.

20. A method for treating a human's body or skin comprising contacting the human's body or skin with a personal care composition according to claim 1.
